(19) United States
(12) Patent Application Publication
(10) Pub. No.: US 2007/0010709 A1
(43) Pub. Date: Jan. 11, 2007

(54) Endoscopy capsule

(76) Inventor: Johannes Reinschke, Nurnberg (DE)

Correspondence Address: HARNESS, DICKEY & PIERCE, P.L.C., P.O.BOX 8910, RESTON, VA, 20195, US

(21) Appl. No.: 11/481,844

(22) Filed: Jul. 7, 2006

(30) Foreign Application Priority Data
Jul. 8, 2005 (DE) DE102005032 369.3

(51) Int. Cl. A61B 1/00; A61M 29/00

(52) U.S. Cl. 600/116; 606/191

(57) ABSTRACT

An endoscopy capsule includes at least one magnetic element, interacting with an externally produced magnetic field, for the magnetic navigation of the endoscopy capsule. At least one gas reservoir is provided, via which at least one balloon is inflateable.

### PRIORITY STATEMENT

The present application hereby claims priority under 35 U.S.C. §119 on German patent application number DE 10 2005 032 369.3 filed Jul. 8, 2005, the entire contents of which is hereby incorporated herein by reference.

### FIELD

The invention generally relates to an endoscopy capsule. For example, it may relate to one including at least one magnetic element, interacting with an external magnetic field, for the magnetic navigation of the endoscopy capsule.

### BACKGROUND

An endoscopy capsule that can be navigated via an external magnetic field is used for recording images from the interior of cavities of different size, primarily the gastrointestinal tract. An advantage of such an endoscopy capsule is that it is navigated solely via the external magnetic field, by contrast with conventional endoscope catheters, which are to be moved from the outside via the guide wire.

The endoscopy capsule is swallowed by the patient and migrates through the gastrointestinal tract under the control of the external magnetic field that interacts with the magnetic element integrated on the capsule side. Cavities of different size occur in the gastrointestinal tract. The small intestine is evacuated and collapsed, but can, like the esophagus, easily be expanded to an inside diameter of from 10-15 mm.

The large intestine can have a substantially larger inside diameter, the largest lumen being located in the stomach. When the endoscopy capsule that is to be navigated magnetically is of smaller geometric dimensions (it is generally of cylindrical design) than the lumen of the hollow organ surrounding it, it is then possible for the capsule to float without making contact, that is to say it is left to itself in this state and can be unstable in more than one dimension with reference to its 6D position (centroid and alignment), and would thus sway arbitrarily were no external magnetic navigation force to be applied. However, if it is guided laterally by the hollow organ, only a defined movement path obtains.

With reference to the required capsule position sensory system and regulation technique, the magnetic capsule navigation is substantially simpler when only a touching movement is possible, that is to say when the capsule is guided laterally by the hollow organ. In such cases, mechanical manipulations that can be carried out via the capsule such as, for example, the deployment of biopsy forceps or of a clip or the like are also capable of being performed in a much easier and accurate fashion.

These circumstances enable an expedient use of an endoscopy capsule only in those cavities in which the capsule is guided by the organ.

### SUMMARY

At least one embodiment of the invention specifies an endoscopy capsule that can be reliably guided and manipulated, even in cavities that are of larger diameter than the endoscopy capsule.

According to at least one embodiment of the invention, in the case of an endoscopy capsule, at least one gas reservoir is provided via which at least one balloon can be inflated.

The integrated gas reservoir according to at least one embodiment of the invention renders it possible, if required, to inflate a balloon on the capsule side via which the diameter of the endoscopy capsule can substantially enlarge. It is thereby possible, when required, to adapt the capsule diameter to the hollow organ, for example to the diameter thereof, such that organ contact. Thus, a contacting capsule movement is possible, even in the region of the hollow organ.

Consequently, the movement of an endoscopy capsule can be controlled by the externally produced magnetic field in a fashion guided by the organ wall. Possible functions such as the deployment of a biopsy forceps or the like are easily possible since the endoscopy capsule can be held in position by the external magnetic field and can be reliably fixed and provided with reaction support via the balloon bearing against the organ wall. A further advantage is that the capsule can also be fixed at any desired point in the hollow organ via the inflated balloon, that is to say can be held fixed in position.

Since many endoscopic procedures require a number of medical measures to be carried out, such as, for example, the recording of images, a biopsy, setting a clip or a drug delivery, it is frequently necessary to use a number of endoscopy capsules and to navigate them through the gastrointestinal tract simultaneously in a magnetically controlled fashion. The magnetic navigation of the capsules is, however, possible or expedient only sequentially, since they are generally situated relatively close to one another. The inflated balloon now permits one or more of the capsules to be fixed such that another capsule can be specifically moved and navigated via the external magnetic field. The fixing is free from injury and can be done virtually at any desired point.

A further advantage of the inflated balloon, and thus of the expansion in diameter, is that organ regions can also be expanded locally such that any constricted points such as stenoses can be treated, or it is directly possible to look more accurately at the intestinal mucosa which, without expansion, has contracted or collapsed. It is also possible thereby to place a stent that is arranged on the capsule.

The endoscopy capsule has a gas reservoir in which liquid gas is contained in a highly compressed form, which gas reservoir can be triggered via a central capsule-side control device that communicates with an external control or operating device in a wireless fashion via a radio device, such that the balloon can be inflated if required. It is expedient, in at least one embodiment, to provide between the gas reservoir and the balloon a valve that can be triggered via the capsule-side control device. Venting the balloon gas, if this is required, is preferably done likewise via an electric valve that can be triggered via the control device, the gas being vented into the capsule environment. It is expedient, in at least one embodiment, for a gas line to lead from the balloon to at least one outlet opening, in or on which gas line the valve is arranged.

The gas accommodated in compressed form in the gas reservoir can expediently be used not only to inflate a balloon, but also for cleaning purposes. Since the inner intestinal wall is not always entirely clean at every point, even in the evacuated state (with the aid of the wash which is customary before a gastroenterological examination), it would be expedient to have the possibility of cleaning on site. To this end, according to at least one embodiment of the invention, the gas reservoir is connected to one or to at least one outlet opening via a gas line, which outlet opening is preferably arranged adjacent to a viewing window of an image recording device, or other sensor device, for example a contactivity sensor, provided on the capsule side, in or on which gas line a valve that can be triggered via the control device is arranged. It is thus possible, if required, to clean the intestinal wall or a part of the capsule surface in the region of the viewing window via a short gas burst, that is to say minor occurrences of contamination can be blown away in this manner.

A further advantageous refinement of at least one embodiment of the invention provides that two or more balloons are provided separately via the gas reservoir to which they are coupled via separate valves that can be triggered via the control device. These balloons can exhibit different geometries and/or different aims of use. For example, one balloon can be a “stomach balloon” that can be inflated to a large volume and via which the capsule exterior becomes quasispherical and fills up the majority of the stomach volume such that the capsule still need only be rotated but can no longer be moved linearly, in order to scan the entire mucosa of the stomach or the stomach wall with the aid of a camera internal to the capsule. A second balloon can, for example, be a “large intestine balloon” that primarily expands the diameter of the capsule such that the latter can bear against the wall of the large intestine. A third balloon can be provided, for example, for fixing, this balloon having a relatively short dimension of length but being capable of inflation to a relatively great extent such that it is possible to achieve a very clear enlargement of diameter. It is, for example, also conceivable to use one or the other balloon to deliver a medically active substance, or else to choke off an injured region for the purpose of hemostatis, or the like.

The balloon or the balloons expediently include an elastic material such that they bear closely and directly against the capsule in the uninflated state. The elasticity simultaneously enables a simple inflation and achievement of the desired balloon geometries. It is expediently possible for the balloon or the balloons also to consist of a nonlinear elastic material. The latter can be inflated elastically as far as a defined shape, but with rising pressure this shape is retained. This is possible, for example, by way of a balloon material that has two elastic plastic membranes between which there is a fiber layer that defines the maximum inflatable geometry. The elastic material could be further inflated without this fiber layer.

The use of elastic material further renders it possible to arrange a balloon in a fashion integrated in another balloon. If, for example, there is arranged in an outer, large “stomach balloon” which can be inflated very far with the formation of a spherical shape, for example, a narrow, short fixing balloon or the like, which can likewise, however, be inflated relatively far, it is possible to inflate only the fixing balloon, upon which the outer “stomach balloon” is likewise slightly widened, but is not itself actively inflated. By virtue of this balloon integration, it is possible to provide different types of balloon in common on the capsule, and to trigger them separately with ease.

The endoscopy capsule itself is expediently of circularly cylindrical design. This is the optimum shape, since the capsule must be swallowed by the patient and migrates through the elongated hollow organs of the gastrointestinal tract. In the inflated state, a balloon itself can expand the cylindrical shape symmetrically while enlarging the diameter.

Alternatively, it is also conceivable, if required, to use an asymmetrically inflatable balloon that varies the cylindrical shape asymmetrically in the inflated state. For example, it can be expedient in the case of a capsule design where the customary image recording unit or video recording unit is not aligned with its imaging axis on the longitudinal axis of the capsule, but at an angle thereto, since it is possible via the asymmetrically inflatable balloon for the capsule to be pressed with the camera side against the intestinal wall such that the latter is dilated and any folds or the like can thereby be opened.

### BRIEF DESCRIPTION OF THE DRAWINGS

Further advantages, features and details of the invention emerge from the example embodiments described below, as well as with the aid of the drawings, in which:

FIG. 1 shows a schematic of an endoscopy capsule according to the invention in a first embodiment,

FIG. 2 shows a capsule from FIG. 1 in a view rotated by 90°,

FIG. 3 shows an endoscopy capsule according to the invention in a second embodiment, with a first inflated outer balloon, and

FIG. 4 shows the capsule from FIG. 3 with a second balloon, inflated here, that is integrated in the first outer balloon.

### DETAILED DESCRIPTION OF THE EXAMPLE EMBODIMENTS

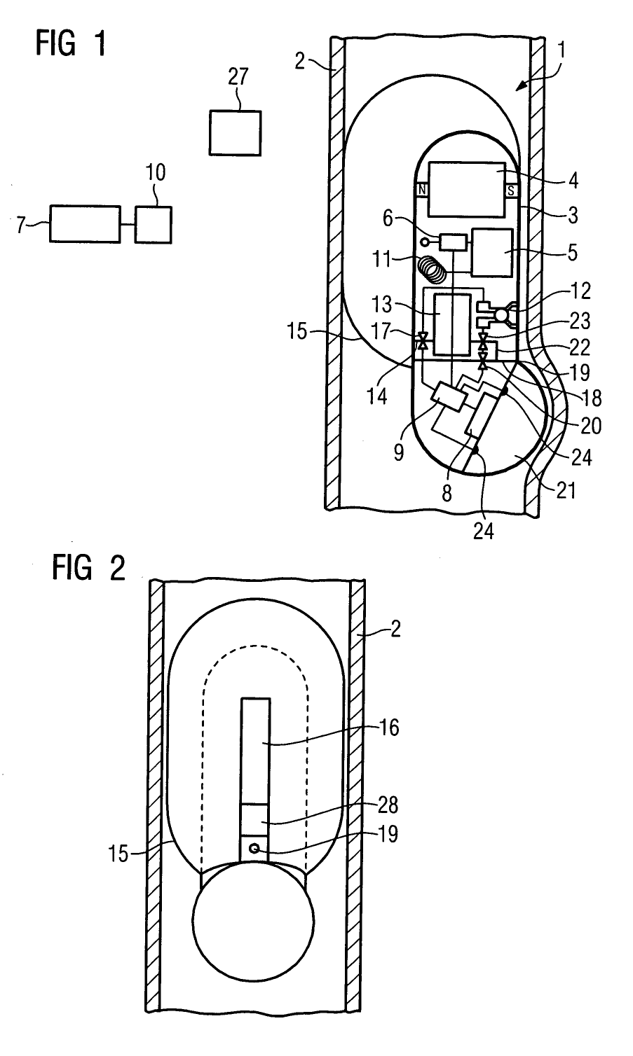

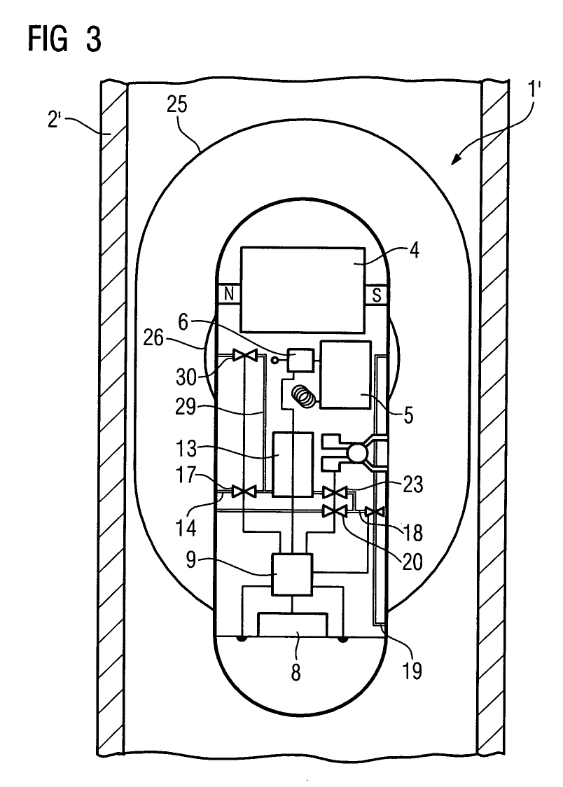

FIGS. 1 and 2 show an endoscopy capsule 1 according to an embodiment of the invention that is shown in the interior of a hollow organ 2, here the intestine. The endoscopy capsule 1 has a capsule housing 3 that is substantially cylindrical. It includes a plastic material. Arranged in the interior, firstly, is a magnetic element that interacts with a magnetic field produced via a magnetic field device 27 arranged externally to the patient being examined. The magnetic element 4 can be, for example, a ferromagnet, and can also be a ferromagnetic material that can be magnetized in an external magnetic field, or be a coil through which current flows. Forces and torques that lead to a defined navigation and movement of the capsule can be specifically exerted on the endescopy capsule owing to the interaction of this magnetic element 4 with the magnetic fields produced by the external magnet system (coil system).

Furthermore, a battery 5 is integrated on the capsule side, as is a radio frequency transceiver 6 that serves as transmitting and receiving antenna, and via which it is possible to communicate with an external operating device 7 via which the controllable elements (such as biopsy forceps, electric valves etc) of the endoscopy capsule 1 are operated. Furthermore, image signals or other measurement signals that are picked up via an image recording device 8, here an electronic camera (CMOS/CCD), or another capsule-side sensor and are passed to the radio frequency transceiver 6 via an integrated control device 9 that controls all the elements internal to the capsule are transmitted to the operating device 7, which can display them on a monitor 10, via this radio frequency transceiver 6.

Integrated, furthermore, is a position sensor 11 that serves the purpose of acquiring the exact position of the capsule in the coordinate system of the external magnetic field production device 27. It is possible via biopsy forceps 12, likewise integrated, to take tissue samples or the like if required. The biopsy forceps 12 can be deployed for this purpose via a biopsy hatch 28.

Further integrated in the capsule interior is a gas reservoir 13 that is connected via a gas line 14 to a balloon 15, arranged on the outside of the capsule, that is illustrated here in an inflated fashion in the example shown. The balloon 15 is made from an elastic material, which is, however, restricted as to its maximum inflatable shape, and arranged such that the capsule shape can be varied asymmetrically—see FIGS. 1 and 2. The balloon is fixed on a side of the capsule (on the right, in FIG. 1), a piece of capsule housing remaining free in the middle region 16.

The gas reservoir 13 is coupled to the balloon 15 via an electric valve 17 integrated in the gas line. This electric valve is triggered and opened if required via the control device 9 such that compressed gas flows from the gas reservoir 13 via the gas line 14 into the balloon 15 and inflates the latter. Since the balloon 15 consists of a nonlinear elastic material, that is to say can be inflated only into a defined maximum shape even when the inflation pressure rises still further, the valve 17 can remain open; it can, however, also be closed again via the control device 9 after a certain time.

Provided for the purpose of venting the air from the balloon 15 is a second gas line 18 that leads to a gas exit opening 19 on the outside of the capsule, and which is connected to the balloon interior. Via an integrated electric valve 20 that can likewise be triggered via the control device 9, the gas line 18 can, if required, be opened and the air can consequently be let out of the balloon. The latter collapses and, having been elastically dilated, lies tightly in the non-inflated shape against the capsule body 3. A smooth surface is thereby formed with the least possible friction in relation to the intestinal mucosa.

It may be seen that the gas outlet opening in the capsule region 16 not covered by the balloon is arranged and positioned such that the escaping gas jet flows directly onto the intestinal wall such that the latter can be cleaned of any possible contaminants. Again, since the viewing window 21 via which the image recording device 8 is enclosed is directly adjacent to the gas outlet opening, this window is likewise cleaned thereby.

Also provided is a third gas line 22 that connects the gas reservoir 13 to the gas line 18, into which gas line 22 a further electrically triggerable valve 23 is switched that can likewise be actuated via the control device 9. It is possible thereby to output a gas burst via the gas outlet opening 19 by opening the electric valve 23 independently of the venting of the inflation air of the balloon 15.

Also illustrated are two illumination devices in the form of small LEDs 24, that are arranged adjacent to the image recording device 8 and are used to illuminate the surrounding organ volume for the purpose of image recording. The LEDs 24 are likewise driven by the control device 9. The power supply of all the electric consumers is provided via the battery 5, the individual elements being triggered/driven via the wireless communication with the external operating device 7, as set forth.

It may be seen that the external geometric shape of the capsule can be adapted by remote wireless control to the surrounding hollow organ so as to enable a movement guided via the intestinal wall 2. The endoscopy capsule 1 bears against the intestinal wall via the balloon as well as the capsule housing and the window 21. Swaying or similar movement by external capsule shapes adapted in such a way is impossible. The navigation takes place via the magnetic interaction, from the outside, unlike when using an endoscope, and this is substantially more pleasant for the patient.

It is possible at just about any desired site to clean the intestinal wall or parts of the capsule surface from relatively small contaminants via the gas outlet opening 19. Again, the endoscopy capsule can be fixed via the inflatable balloon at sites that are somewhat smaller in diameter than the maximum capsule diameter in the inflated state. It is also possible to expand the hollow organ in this region, and to set, for example, a stent or a chip (neither of which is shown here any further). Also possible via the balloon is targeted drug delivery, for which purpose it is coated, for example, on the outside with an appropriate device/method. It is also possible to use the inflated balloon to choke off hemorrhages.

FIG. 3 shows an inventive endoscopy capsule 1′ in the case of which all the elements described with reference to FIG. 1 are present (although no more detail is given on this). By contrast with the capsule from FIG. 1, however, the image recording device is here aligned in the longitudinal capsule direction, that is to say not tilted as shown in FIG. 1.

In the example embodiment shown, two balloons 25, 26 are provided on the endoscopy capsule 1 in accordance with FIG. 3. The balloon 25 is a large outer balloon that can be inflated to a maximum, defined volume, and via which it is possible to adapt substantially to the diameter of the intestine or the like for the purpose of guidance by contact. This state is illustrated in FIG. 3.

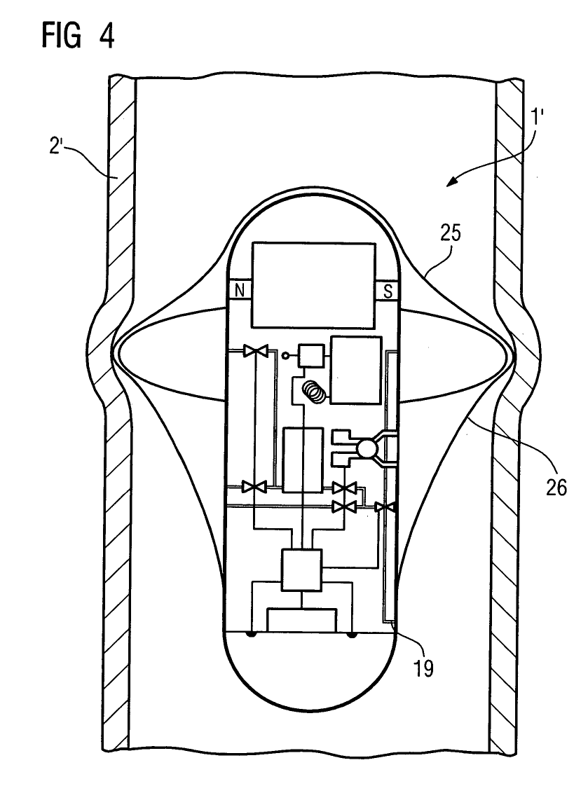

FIG. 4 shows the case where the endoscopy capsule 1′ is to be fixed at a specific site. The outer balloon 25 is here not inflated, or air has been vented from it in advance. The inner balloon 26 is now inflated for the purpose of fixing. Seen in the longitudinal direction of the capsule, the balloon is relatively narrow, but can be inflated to a very large diameter. To this end, it is connected to the gas reservoir 13 via a line 29 with an integrated electric valve 30 that can be triggered via the control device 9.

Via a further line (not shown here for reasons of clarity), with a valve, the gas can also be vented from the balloon 26 and, for example, be output to the outside via the line 18 in a way similar to that described with reference to the balloon 15 in FIG. 1, and is likewise possible in the case of the balloon 25 in FIG. 3. It may be seen that it deforms the intestinal wall 2′ somewhat such that the capsule 1′ is fixed overall on the intestinal wall at the desired position. This renders it possible to undertake appropriate video observation in the fixed state via the image recording device, or else, if required, to use a biopsy tool to remove a sample, or the like, or else merely to fix this capsule 1′ in order to hold a second capsule, which is arranged in parallel in the intestine, via the external navigation device.

The gas pressure that can be produced via the gas reservoir 13 should be dimensioned such that the respective balloon can be completely inflated in any case, that is to say it can be inflated into a defined final shape that is not deformed even when there is slight pressure on the intestinal wall, that is to say when the capsule slider along the latter. The capsule or the inflated balloon then acts like a rigid body, and thus cannot be pressed in, something which is expedient for capsule navigation with as little force required as possible. The gas volume itself should be dimensioned such that the balloon or balloons can be reversibly inflated repeatedly.

Further, elements and/or features of different example embodiments may be combined with each other and/or substituted for each other within the scope of this disclosure and appended claims.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An endoscopy capsule, comprising:
at least one magnetic element, to interact with an externally produced magnetic field, for the magnetic navigation of the endoscopy capsule; and
at least one gas reservoir, which at least one balloon is inflatable.

2. The endoscopy capsule as claimed in claim 1, further comprising a valve, triggerable via a control device provided on the capsule side and able to communicate in a wireless fashion with an external operating device, arranged between the gas reservoir and the balloon.

3. The endoscopy capsule as claimed in claim 2, wherein the balloon is assigned an electric valve, triggerable via the control device, to vent the gas into the capsule environment.

4. The endoscopy capsule as claimed in claim 3, wherein a gas line leads from the balloon to at least one outlet opening, the valve being arranged at least one of in and on the gas line.

5. The endoscopy capsule as claimed in claim 1, wherein the gas reservoir is connected via a gas line to at least one outlet opening, at least one of in and on which gas line a valve, triggerable via the control device, is arranged.

6. The endoscopy capsule as claimed in claim 1, wherein at least two balloons are provided separately via the gas reservoir, to which they are coupled via separate valves that are triggerable via the control device.

7. The endoscopy capsule as claimed in claim 1, wherein the balloon includes an elastic material.

8. The endoscopy capsule as claimed in claim 7, wherein the balloon includes a nonlinearly elastic material.

9. The endoscopy capsule as claimed in claim 6, wherein a first balloon is integrated in a second balloon.

10. The endoscopy capsule as claimed in claim 1, wherein the endoscopy capsule is of circularly cylindrical design, in the inflated state, a balloon at least one of expanding the cylindrical shape symmetrically with enlargement of the diameter, and being arranged asymmetrically and varying the cylindrical shape asymmetrically in the inflated state.

11. The endoscopy capsule as claimed in claim 5, wherein the gas reservoir is connected via a gas line to at least one outlet opening, arranged adjacent to a viewing window of at least one of an image recording device, provided on the capsule, and a sensor device.

12. The endoscopy capsule as claimed in claim 2, wherein the gas reservoir is connected via a gas line to at least one outlet opening, at least one of in and on which gas line a valve, triggerable via the control device, is arranged.

13. The endoscopy capsule as claimed in claim 2, wherein at least two balloons are provided separately via the gas reservoir, to which they are coupled via separate valves that are triggerable via the control device.

14. The endoscopy capsule as claimed in claim 3, wherein the gas reservoir is connected via a gas line to at least one outlet opening, at least one of in and on which gas line a valve, triggerable via the control device, is arranged.

15. The endoscopy capsule as claimed in claim 3, wherein at least two balloons are provided separately via the gas reservoir, to which they are coupled via separate valves that are triggerable via the control device.

16. The endoscopy capsule as claimed in claim 2, wherein the balloon includes an elastic material.

17. The endoscopy capsule as claimed in claim 3, wherein the balloon includes an elastic material.

18. The endoscopy capsule as claimed in claim 6, wherein the balloons include an elastic material.

19. The endoscopy capsule as claimed in claim 7, wherein a first balloon is integrated in a second balloon.

20. The endoscopy capsule as claimed in claim 8, wherein a first balloon is integrated in a second balloon.
